(19) United States
(12) Patent Application Publication
(10) Pub. No.: US 2001/0020002 A1
(43) Pub. Date: Sep. 6, 2001

(54) Androgen activity antagonists as therapies for anorexia, anorexia nervosa and disorders characterized by a pathologically underweight condition

(75) Inventors: Seth Lederman, New York, NY (US); Donald W. Landry, New York, NY (US)

Correspondence Address: FISH & NEAVE, 1251 AVENUE OF THE AMERICAS, 50TH FLOOR, NEW YORK, NY, 10020-1105, US

(73) Assignee: VELA PHARMACEUTICALS INC.

(21) Appl. No.: 09/834,468

(22) Filed: Apr. 13, 2001

Related U.S. Application Data
(63) Continuation of application No. 08/919,301, filed on Aug. 28, 1997, now abandoned.

(51) Int. Cl. A61K 38/17; A61K 31/575; A61K 31/496

(52) U.S. Cl. 514/2; 514/65; 514/253.08; 514/254.07; 514/284; 514/179

(57) ABSTRACT

The present invention relates to the treatment of conditions characterized by loss of appetite (anorexia) and/or pathological weight loss by administering a therapeutically effective amount of an agent that modulates androgen activity. The present invention further relates to the treatment and prevention of anorexia nervosa with such agents.

### 1. FIELD OF THE INVENTION

The present invention relates to the treatment of conditions characterized by loss of appetite (anorexia) and/or pathological weight loss by administering a therapeutically effective amount of an agent that modulates androgen activity. The present invention further relates to the treatment and prevention of anorexia nervosa with such agents.

### 2. BACKGROUND OF THE INVENTION

### 2.1 Loss of Appetite and Conditions Characterized by a Pathological Weight Loss

“Anorexia” is a general term meaning loss of appetite. It is a symptom of many diseases which may lead to malnourishment. Certain types of diseases and disorders are marked by a severe weight loss resulting in a pathologically underweight state. Such conditions may be the result of acquired immune deficiency syndrome (AIDS), cancer, substance abuse, substance withdrawal and stress. Anorexia, for instance, is associated with wasting (cachexia) in both AIDS and cancer. Anorexia in AIDS patients may be caused by a variety of known causes such as opportunistic infections which can be treated by conventional therapies. However, anorexia of unknown etiology also occurs, and is therefore more difficult to treat. Von Roenn, J. H., 1994, Drugs, 47(5), 774-83. Cachexia occurs in the majority of cancer patients before death with the most significant sign being anorexia. Bruera, E., 1992, Oncology, 49 (Suppl.) 2, 35-42.

One of the most striking disorders characterized by a pathologically underweight condition is anorexia nervosa (AN). To the extent that anorexia means “loss of appetite,” the name “anorexia nervosa” is something of a misnomer and is inaccurate; typically, individuals with AN do not experience a loss of appetite. AN is more accurately defined as “self-starvation.” AN is a life-threatening disorder which strikes 0.2-1.3 percent of the general population (most commonly in industrialized nations) and primarily affects young women; only about 5-10 percent of AN patients are male. Hobbs, Wendy & Cynda Ann Johnson, American Family Physician, 54(4) 1273 (Sep. 15, 1996). The overall mortality rate in anorexia nervosa is approximately 10% of individuals admitted to university hospitals. American Psychiatric Association's Diagnostic & Statistical Manual of Mental Disorders (4th Ed. 1995).

According to the American Psychiatric Association's Diagnostic & Statistical Manual of Mental Disorders (4th Ed.), § 307.1 (1995), there are four criteria to diagnose AN: (1) severe weight loss in which affected individuals have a body weight less than 85% of expected body weight, (2) an intense fear of weight gain, (3) significant distortion of self-perception of body weight and shape, and (4) amenorrhea (in pre-pubescent females, a delay in the onset of menarche). Determination of expected body weight is made by comparison to the values found in the Metropolitan Life Insurance tables or pediatric growth charts. Alternatively, the ICD-10 Diagnostic Criteria for Research indicates that “underweight” is a body mass index (BMI) of 17.5 kg/m², or less, where BMI is determined as body weight in kg/height² in meters².

AN is a separate and distinct disorder from Bulimia nervosa. Unlike patients with AN, patients with Bulimia nervosa maintain a body weight within a normal range, i.e. bulimics do not suffer from the severe weight loss and distortion of body weight and shape which are central to AN. Furthermore, amenorrhea, a defining characteristic of AN, is not a typical feature of bulimia. Also, unlike AN patients, bulimic patients do not avoid eating. Instead, bulimic patients gorge themselves and force themselves to vomit afterwards and/or abuse laxatives, an activity known as “binging and purging.” Bulimia remains a separate and distinct disorder from AN.

Anorexia is difficult to treat in general. The available treatments for AIDS-related anorexia include appetite stimulants. Von Roenn, J. H., 1994, Drugs, 47(5), 774-83. The appetite stimulants in use as therapies for anorexia include megastrol acetate, adrenocorticoids such as prednisolone and dexamethasone, and cyproheptidine. Loprinzi, C. L., et al., 1992, Drugs, 43(4) 499-506. There have also been experimental results suggesting the use of fenfluramine (a serotonergic drug) and neuropeptide Y as appetite stimulants. Rogers, P., 1991, Peptides, 12(2) 251-5. Recently, a derivative of marijuana, delta-9-tetrahydrocannabinol (THC), has been used as an appetite stimulant for patients with AIDS-related anorexia, and for cancer patients to curb the nausea and vomiting related to some forms of chemotherapy. However, new therapies to treat the underlying causes of anorexia are highly desirable, as conventional therapies have demonstrated limited success.

AN is particularly resistant to treatment, and patients often refuse treatment. The proper treatment for AN is food. Adrienne de Mont, Food Crisis, Chemist & Druggist, Sep. 21, 1996. Although, conventional therapy for severe AN has involved force-feeding through naso-gastric tubes and psychological counseling, current treatment of AN focuses on treating the psychological aspects of the disease. Although tranquilizers, antidepressants and hypnotics are helpful in treating the anxiety, depression and insomnia that often accompany the disorder, these drugs do not treat the underlying disorder. Adrienne de Mont, Food Crisis, Chemist & Druggist, Sep. 21, 1996.

AN must be treated over a long period of time, and relapses are frequent. Recidivism rates in eating disorders may reach ninety-seven percent (The Ethnic Newswatch, Northern California Jewish Bulletin, Softline Information, Inc., Nov. 22, 1996).

Prozac® has helped AN patients from relapsing after recovery, but has not been an efficacious therapeutic agent. Lytle, Health Notes, Orange County Register, May 28, 1997, p E1. Moreover, only a small percentage of patients ever become free of eating disorders, including patients on high dosages of Prozac® (The Ethnic Newswatch, Northern California Jewish Bulletin, Softline Information, Inc., Nov. 22, 1996). According to a recent report, no medication has been found to be effective in treating AN thus far. Amara, A. RN, 59(6), 30 (1996).

### 2.2 Anti-Androgens in General

Many known compounds are used to modulate androgen production, secretion, metabolism or activity. To the Applicant's knowledge, reduction of androgen activity has not been targetted previously for the treatment of anorexia, AN or other disorders featuring pathological weight loss, and none of the following agents has been so used.

Flutamide, nilutamide, and zanoterone are examples of compounds which act as androgen antagonists. Flutamide is currently used to treat advanced prostatic cancer and has been evaluated experimentally as a treatment for hirsutism in women. Moghetti, et al, 1995, Fertility and Sterility 64(3) 511-17. There is also an anecdotal report of the use of flutamide in two patients with bulimia to curb impaired impulse control and depressed mood. Bergman, et al., 1996, Acta Psychiatr. Scand., 94, 137-39.

Finasteride (Proscar®) inhibits 5α-reductase which converts testosterone to 5α-dihydrotestosterone (a more potent androcen with a different spectrum of action). Finasteride, typically, is used to treat symptomatic benign prostatic hyperplasia.

Estrogen therapy, including estradiol and diethylstilbestrol (DES) is used to treat hypoestrogenism, and hypogonadism, to prevent osteoporosis in some patients, and as palliative therapy in advanced metastatic breast cancer and prostate carcinomas.

There is a need for an effective therapy for the treatment of anorexia, AN, and other disorders featuring a pathologically underweight condition. In particular, it is highly desirable to have a treatment for anorexia, AN and disorders characterized by a pathologically underweight condition which alleviates the symptoms of the disorder without either interfering with concomitant treatments or causing side effects.

### 3. SUMMARY OF THE INVENTION

The present invention provides a method for treating conditions characterized by loss of appetite and/or pathological weight loss by administering a therapeutically effective amount of an agent that modulates androgen activity. As used herein, the term “modulate” refers to an alteration of androgen activity, through the action of an androgen antagonist, which tends to result in a reduction of androgen or the effect of the androgen. Treatment and prevention of AN with such agents is also provided. As used herein, the term “therapeutically effective amount” generally refers to a dosage sufficient to modulate androgen activity in the body, which depends in part on the specific agent used, and the patient being treated, as decribed in detail herein.

The treatment methods of the present invention are based, in part, on the discovery that excess androgen activity contributes to anorexia, AN and other disorders which result in a pathologically underweight state in humans. Thus, agents which effectively reduce excess androgen activity in the body, either by reducing androgen synthesis or secretion, promoting androgen metabolism, interfering with androgen/androgen receptor interaction, or androgen receptor signaling are useful for treating anorexia, AN and other disorders which result in a pathologically underweight state.

Suitable agents useful for modulating androgen activity include, but are not limited to androgen receptor antagonists; 5α-reductase inhibitors; estrogen agonists; promoters of androgen metabolism; HMG-CoA reductase inhibitors; agents which down-modulate androgen receptors; recombinant DNA and RNA products, recombinant viruses and other gene therapy strategies.

Androgen receptor antagonists useful in the present invention include, but are not limited to flutamide, zanoterone, nilutamide, cimetidine, casodex, pyrethroids (including but not limited to pyrethrins, bioallethrin, fenvalerate, fenothrin, fluvalinate, permethrin, and resmethrin), Anandron (RU 23908), and other new non-steroidal anti-androgens (e.g., RU 59063, RU 56187; see, for example, Teutsch et al., 1994, Journal of Steriod Biochemistry & Molecular Biology 48:111).

Inhibitors of 5α-reductase suitable for use in the present invention include, but are not limited to finasteride, FCE 28260, FE 28175, and FE 27837. See for example, di Salle et al., 1994, Journal of Steroid Biochemistry & Molecular Biology 49:289.

Estrogen agonists useful in the present invention include, but are not limited to diethylstilbestrol (DES), estradiol, 17-epistriol, and 6-methylene-4-pregnene-3,20-dione.

Androgen antagonists suitable for use in the present invention include, but are not limited to spironolactone, ketoconazole, ciprofloxacin and epitestosterone.

Agents that promote androgen metabolism suitable for use in the present invention include, but are not limited to aromatase.

HMG-CoA reductase inhibitors suitable for use in the present invention include, but are not limited to lovastatin and simvistatin.

Molecular biological products suitable for use in the present invention include, but are not limited to recombinant DNA and RNA products, recombinant proteins, recombinant mutant proteins, recombinant fusion proteins, recombinant viruses, and recombinant vectors to deliver gene therapy.

Genes and gene products suitable for use in the present invention include, but are not limited to the neutral amino acid transporters, tryptophan hydroxylase, aromatase, and abnormal androgen receptors.

The administration of agents that reduce androgens to patients with AN or other disorder characterized by anorexia and a pathologically underweight condition, is a novel approach to treatment of these intractable diseases. The invention discloses for the first time, a pharmacological treatment for AN, anorexia, and disorders featuring a pathologically underweight condition by reducing androgen activity in affected individuals.

Many known drugs and therapies are effective in reducing androgen activity and are useful in the method of the present invention. Effective reduction of excess androgen activity may be accomplished by reducing androgen synthesis and/or secretion, promoting androgen metabolism, interfering with androgen/androgen receptor interaction and/or interfering with androgen receptor signaling.

### 4. DETAILED DESCRIPTION OF THE INVENTION

The present invention encompasses a method of treating anorexia, anorexia nervosa, and other conditions featuring a pathologically underweight condition by administering a therapeutically effective amount of an anti-androgen to modulate the effect of excess androgen or androgen activity. The present invention further relates to prevention of AN with anti-androgens. For the sake of convenience, and not intended as limiting, anti-androgen, as used herein, includes but is not limited to, androgen synthesis inhibitors, promoters of androgen metabolism, agents interfering with the binding of natural androgen to androgen receptor (and agents interfering with androgen receptor signaling.

Anorexia nervosa has been linked with a myriad of biochemical abnormalities but heretofore none were considered responsible for the pathogenesis of the psychological abnormality underlying anorexia nervosa. An increase in androgen activity, whether due to increased androgen levels, or an increased effectiveness of normal levels of androgen, was previously considered an epiphenomenon of the anorectic state. However, in the course of inquiring into the behavioral effects of androgens, it was unexpectedly found that increased androgen activity plays an integral role in the pathogenesis of anorexia nervosa.

It is known that sex steroids control certain psychological states and are dysregulated during hormonal surges of puberty (prior to and after menarche). In females, sex steroid changes, particularly an increase in androgens, can be associated with hormone-dependent, psychologically dysfunctional states, such as AN. The food avoidance behavior that characterizes AN, thought to be either induced by or maintained by altered self-perception of body image, is actually driven by and dependent on stimulation of androgen receptors in the central nervous system (CNS). Phylogenetically primitive CNS control of food seeking and avoidance is androgen-stimulated. In this way, eating behaviors are similar to other androgen-dependent behavioral and psychological states such as aggression, attack and predatory behaviors. The androgen receptor system that controls this response is the phylogenetically primitive amygdala (or hippocampus).

It is known that sex steroids exert potent effects on behavior. It has not been shown previously that androgens exert an important effect on the food avoidance behavior of the psychological state underlying anorexia nervosa. However, androgen activity is stimulated by life stresses. In the context of AN, examples of such life stresses include, but are not limited to, the separation or divorce of parents and the novelty of leaving home for college. Therefore, individuals who are susceptible to AN, by virtue of being peri-pubescent, are driven to AN by increased androgen activity as well as other factors that contribute to pathology in AN. In this self-reinforcing manner, the excess androgen activity leads to decompensation. Androgen antagonists can break the decompensatory cycle, inhibit physical and biochemical manifestations of AN, maintain the non-anorexia nervosa state in recovering or susceptible individuals and prevent relapses of AN.

Although the invention focuses on AN in female adolescents and young adults, it also describes how excess androgen activity can induce or exacerbate food avoidance and underweight states in females of other ages and in males. Anorexia nervosa in males is also treated by anti-androgens. In addition, this invention has ramifications for understanding and treating the alteration in body weight in other conditions, such as drug addiction, AIDS and cachexia and emaciation related to other states.

Without being limited by theory, the invention is based, in part, on the discovery that excess androgen activity is implicated as the cause of anorexia and AN. This increased androgen activity could reflect increased androgen levels or increased androgen sensitivity. Increased levels of serum testosterone are associated with AN and may precipitate or maintain AN. Although elevated levels of testosterone have not been uniformly observed in AN patients, the reason may be that serum testosterone is a poor measure of testosterone activity in tissues. For example, studies in rats have revealed that tissue levels of ovarian and adrenal steroids do not correlate with levels in plasma.

Whether from absolutely increased androgen levels or abnormal sensitivity to otherwise normal levels, there are several functional measures of androgen activity in female AN patients that are consistent with increased androgen activity. First, high testosterone induces psychological manifestations of AN. Second, in AN, both baseline and GnRH-induced levels of LH are suppressed to a greater extent than FSH levels. Testosterone is known to act through a negative feedback loop (on hypothalamus and anterior pituatary) to suppress LH to a much greater extent than FSH, and in fact, testosterone can potentiate FSH breakthrough in pituitary suppression by lupron treatment. Third, in AN, ACTH-stimulated adrenal androgen secretion is impaired, which can occur after androgen stimulation.

The present invention uses known compounds which modulate the activity of androgens in the patients requiring treatment. The compounds which may be used according to the present invention include, but are not limited to the following:

Inhibitors of Androgen/Androgen Receptor Interaction

flutamide

zanoterone

nilutamide

cimetidine

casodex

pyrethroids

andaron

RU 59063

RU 56187

5α-reductase inhibitors

Finasteride

Androgen antagonists

epitestosterone

spironolactone

ketoconazole

ciprofloxacin

estrone

Estrogen agonists

diethylstilbestrol (DES)

estradiol

17-epistriol

6-methylene-4-pregnene-3,20-dione

HMG-CoA reductase inhibitors

lovastatin

simvistatin

Promoters of Androgen Metabolism

aromatase

Other agents that affect androgen activity

L-tryptophan

Tryptophan hydroxylase, and

Selective inhibitors for valine, leucine and isoleucine to the amino acid transporter.

As used herein, the term “androgen” comprises the sex steroids; testosterone, dihydrotestosterone, DHEA, and diepiandrostenone.

Further as used herein “activity” comprises the signaling of a ligand/receptor system and includes the bio-availability of ligand, the ability of ligand to interact with receptor, the number and function of the receptors, and the ability of receptor signals to mediate biological effects. As used herein, “androgen activity” comprises the signaling of the androgen/androgen receptor system and includes bio-availability of androgens, the ability of androgens to interact with androgen receptors, the number and signaling of androgen receptors and the ability of androgen receptor signals to mediate biological effects.

Further as used herein, “agent” comprises peptides, peptide analogues, small organic molecules, recombinant peptides, DNA or RNA constructs, or viruses.

Further, as used herein, “anti-androgen” is an agent that reduces androgen synthesis, promotes androgen metabolism, modulates androgen/androgen receptor interaction, or androgen/androgen receptor signaling.

The present invention encompasses a method of treating anorexia, anorexia nervosa, and other conditions characterized by a pathologically underweight condition by administering a therapeutically effective amount of an anti-androgen, said amount being sufficient to alleviate anorexia, anorexia nervosa or other disorders featuring a pathologically underweight state. Preventing AN is also contemplated. The anti-androgens of the present invention include hormonal and non-hormonal compounds, products prepared by recombinant technology, as well as gene therapy strategies.

### 4.1 Drug Dosages

In one embodiment of the invention, patients with anorexia or AN, or other disorder featuring a pathological weight loss, are treated with a therapeutically effective amount of an agent that interferes with androgen/androgen receptor interaction, i.e., a dose sufficient to reduce androgen activity in the body. Nilutamide, flutamide, zanoterone, cyproterone acetate or casodex is administered to reduce androgen activity in the body.

Nilutamide has a half-life of 40 hours, and as such, when it is used in the method according to the present invention, 100 mg is administered three times a day (total of 300 mg/day). Alternatively, a single daily dose of up to 300 mg nilutamide may be administered.

Flutamide has a much shorter half-life (5.2 hours). When flutamide is selected as the anti-androgen according to the present invention, a total of up to 750 mg/day distributed over 3 dosages may be employed. A range of 375-500 mg/day is preferred provided it is effective in reducing androgen activity in the individual patient. Flutamide treatment is the most preferred embodiment of the invention.

Zanoterone is administered at a dosage of 100-800 mg as an oral tablet once per day.

Casodex is generally administered in dosages of 50 mg/day. This dosage may be increased or decreased to optimize therapuetic effect as determined by the physician.

Cyproterone acetate may be administered orally or by an intramuscular injection. Dosage may range between 100 and 300 mg/day depending on efficacy in the individual patient. Of the agents which interfere with androgen/androgen receptor interaction, flutamide, nilutamide and zanoterone are preferred over cyproterone acetate; and flutamide is the most preferred.

In another embodiment of the invention, patients with anorexia or AN, or other disorder featuring a pathological weight loss, are treated with a therapeutically effective amount of a 5α-reductase inhibitor, i.e., a dose sufficient to reduce androgen activity in the body. 5α-reductase prevents the conversion of testosterone to 5α-dihydrotestosterone (DHT) and effectively reduces DHT activity in the body. For example, finasteride (Proscar®) is administered, i.e., a dose sufficient to reduce androgen activity in the body. Typically, finasteride tablets are administered orally at a single dosage of 10 mg/day. Finasteride therapy is a preferred embodiment of the invention.

In another embodiment of the invention, patients with anorexia or AN, or other disorder featuring a pathological weight loss, are treated with a therapeutically effective amount of an agent that reduces androgen production or increases androgen metabolism, i.e., a dose sufficient to reduce androgen activity in the body. For instance, many known drugs used for purposes other than to reduce androgen activity also have an inhibitory effect on androgen synthesis. For example, spironolactone (Aldatazide®) is currently used in primary aldosteronism (including adrenal adenomas); for edematous disease due to congestive heart failure, cirrhosis, and nephrotic syndrome; and for essential hypertension and hypokalemia. However, although not currently used for reducing testosterone synthesis, spironolactone effectively reduces androgen synthesis. Spironolactone is provided in tablet form and is administered at the lowest possible effective dose. Dosages may range from 100 to 400 mg/day as a single or divided dose.

Ketoconazole (Nizoral®) is an anti-fungal imidazole that also has an inhibitory effect on androgen synthesis. Ketoconazole is provided in tablet form and dosages may range from 200 mg to 400 mg.

Ciprofloxacin (Cipro® and Cipro I.V.®) are antibacterials which also have an inhibitory effect on androgen synthesis. Ciprofloxacin may be administered in tablet form or as an injectable. Cipro® is provided in 250, 500 mg and 750 mg doses. Typically, Cipro® is provided at dosages of 250 mg or 500 mg twice a day, every twelve hours.

Cipro I.V.® is administered as a 60 minute infusion (provided as a 2% premixed solution of 1-2 mg/ml)

HMG-CoA reductase inhibitors are currently used to reduce cholesterol. Since androgens are derived from cholesterol, such drugs also have the effect of lowering androgen levels. For example, lovastatin (Mevocor®) and simvastatin (Zocor®) may be used to reduce androgen synthesis. Lovastatin is administered in tablet form, as a single, daily dose of 20 mg. Simvastatin also is administered in tablet form as a single, daily dose of 5-10 mg/day.

In another embodiment of the invention, patients with anorexia or AN, or other disorder featuring a pathological weight loss, are treated with a therapeutically effective amount of an agent that reduces androgen production, i.e., a dose sufficient to reduce androgen activity in the body.

Epitestosterone is administered at the lowest possible effective dose based on body weight or surface area, as determined by the physician.

In another embodiment of the invention, patients with anorexia or AN, or other disorder featuring a pathological weight loss, are treated with a therapeutically effective amount of an estrogen agonist, i.e., a dose sufficient to reduce androgen activity in the body by acting as an androgen antagonist. For example Estradiol, which is currently used to treat hypoestrogenism, hypogonadism, symptoms associated with menopause, prevention of osteoporosis as well as for palliative treatment of metastatic breast and prostate cancers, may be used as an androgen antagonist. Estradiol is administered in capsules (Emcyt®) in which estradiol is linked to nornitrogen mustard and metabolized to give estradiol. This oral preparation is given in divided doses of 14 mg/kg/day. Another oral preparation of estradiol is administered in tablet at a dosage of 0.5-2 mg. Estradiol can also be administered as a vaginal cream containing 0.1 mg/g of cream or as a transdermal patch containing 0.05 mg applied twice weekly.

Diethylstilbestrol (DES) may also be administered as an estrogen receptor agonist. DES is administered as an oral preparation from 1-15 mg daily. The referred range is 1-3 mg, most preferred is 1 mg. Also, estrone may be administered as an estrogen receptor agonist. Suitable forms and dosages of estrone are: estrone piperazine administered orally in an amount of 0.35-1 mg daily; estrone plus estrone potassium sulfate injected in amounts of 0.25-1 ml once or twice per week, (1 ml contains estrone 2 mg and estrone potassium sulfate 1 mg).

The magnitude and therapeutic dose of the anti-androgen in the acute or chronic management of the disorders described herein will vary with the severity of the condition to be treated, the type of anti-androgen or combination of anti-androgens selected, and the route of administration. The dose, and perhaps the dose frequency will vary according to the age, body weight and response of the individual patient. In some cases, it may be necessary to use dosages outside the ranges described above, as will be apparent to those skilled in the art. Further, it is noted that the clinician or treating physician will know how and when to interrupt, adjust or terminate therapy in conjunction with the individual patient response.

Any suitable route of administration may be employed for providing the patient with an effective dosage of the selected anti-androgen. For example, oral, rectal, parenteral (subcutaneous, intramuscular, intravenous), transdermal, and the like forms of administration may be employed. Dosage forms include tablets, troches, dispersions, suspensions, solutions, capsules, patches, and the like.

The anti-androgen of the present invention may be administered alone, or optionally, in combination with other therapeutics. Other therapeutics which may be used in conjunction with anti-androgen therapy include but are not limited to the following: birth control methods, such as oral birth control pills, as many of the drugs adversely affect male fetal development; and inhibitors of bone mineral density loss, such as etidronate, as many of the drugs diminish bone density through decreased androgen activity.

The anti-androgens of the present invention include compositions such as suspensions, solutions and elixirs; aerosols or carriers such as starches, sugars, microcrystalline cellulose, diluents, granulating agents, and the like, in cases of oral solid preparations (such as powders, capsules, and tablets).

The method of treating patients with anti-androgens of the present invention includes the administration of anti-androgens using DHA (a fatty acid used to carry attached molecules to the cerebro-spinal fluid), a lipophilic form of the coenzyme system containing nicotinamide (which helps target therapeutics to the brain), receptor-mediated permeabilizer (RMP-7) (which temporarily increases the permeability of the blood-brain barrier), and fusion proteins that aid in penetrating the blood brain barrier (such as nerve growth factor fusion proteins and transthyrotein fusion proteins).

The anti-androgens of the present invention also include molecular biological therapies. Such therapies include the administration of recombinant proteins and/or fusion proteins, and gene therapy using RNA constructs, DNA constructs and/or viruses. Recombinant proteins and fusion proteins are a preferred molecular biological therapy. Recombinant proteins are the most preferred molecular biological therapy. The efficacy of recombinant proteins and/or fusion proteins may be enhanced with the use of various known delivery systems that allow such proteins to cross the cell membrane. Examples of suitable delivery systems are described by Vives et al., 1997, Journal of Biological Chemistry, 272 (25):16010-7, (HIV Tat delivery system); and Schutze-Redelmeir et al., 1996, Journal of Immunology, 157(2):650-5, and Brugidou et al., 1995, Biochemical & Biophysical Research Communications, 214(2):685-93, (Antennapedia delivery system).

### 4.2 Gene Therapy

In a specific embodiment, nucleic acids comprising a sequence encoding aromatase; neutral amino acid transporter; tryptophan hydroxylase; or various inhibitory forms of testosterone receptor (inhibitory testosterone receptor), such as, a partial sequence of the testosterone receptor, or an abnormal sequence for the testosterone receptor or an alternatively spliced version of the testosterone receptor (as a dominant negative form of gene therapy), are used to treat AN, anorexia or pathologically underweight conditions. For example, aromatase catalyzes the conversion of testosterone to estrogen, thereby reducing androgen in the body. Likewise, over expression of aromatase effectively increases androgen metabolism. See, e.g., Macaulay et al., 1994, British J. Cancer 69:77-83. Furthermore, peptide or protein derivatives of these genes may be administered for the treatment of anorexia, AN or pathologically underweight condition by way of gene therapy. Gene therapy refers to therapy performed by the administration of a nucleic acid to a subject. In this embodiment of the invention, the nucleic acid produces its encoded protein that mediates a therapeutic effect sufficient to treat anorexia, AN or disorders featuring a pathologically underweight condition. For example, any of the methods for gene therapy available in the art can be used according to the present invention. An exemplary method is described below, but it will be understood by those skilled in the art that any of the above described gene targets for gene therapy may be substituted.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

A nucleic acid encoding a target protein such as aromatase; neutral amino acid transporter; tryptophan hydroxylase; or a partial sequence of the testosterone receptor, an abnormal sequence for the testosterone receptor or an alternatively spliced version of the testosterone receptor is part of an expression vector that produces the respective target protein in a suitable host. In particular, such a nucleic acid has a promoter operably linked to the nucleic acid sequence coding for the target protein, said promoter being inducible or constitutive, and, optionally, tissue-specific. In another particular embodiment, a nucleic acid molecule is used in which the target protein encoding sequence and any other desired sequences are flanked by regions that promote homologous recombination at a desired site in the genome, thus providing for intrachromosomal expression of the aromatase nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then administered to the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the cell or nucleus, e.g., by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In a specific embodiment, the nucleic acid can be targeted in vivo for cell specific uptake and expression, by targeting a specific receptor (see, e.g., PCT Publications WO92/06180 dated Apr. 16, 1992 (Wu et al.); WO92/22635 dated Dec. 23, 1992 (Wilson et al.); WO92/20316 dated Nov. 26, 1992 (Findeis et al.); WO93/14188 dated Jul. 22, 1993 (Clarke et al.), WO93/20221 dated Oct. 14, 1993 (Young)). In another embodiment, a nucleic acid-ligand complex can be formed in which the ligand comprises a fusogenic viral peptide to disrupt endosomes, allowing the nucleic acid to avoid lysosomal degradation. Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the nucleic acid sequence encoding the target protein is used. For example, a retroviral vector can be used (see Miller et al., 1993, Meth. Enzymol. 217:581-599). These retroviral vectors have been modified to delete retroviral sequences that are not necessary for packaging of the viral genome. Retroviral vectors are maintained in infected cells by integration into genomic sites upon cell division. The nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300). Herpes viruses are other viruses that can also be used.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including, but not limited to, transfection, electroporation, microinjection, infection with a viral vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell.

The resulting recombinant cells can be delivered to a patient by various methods known in the art. In a preferred embodiment, epithelial cells are injected, e.g., subcutaneously. In another embodiment, recombinant skin cells (e.g., keratinocytes) may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

In an embodiment in which recombinant cells are used in gene therapy, the target protein encoding sequence is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

### 4.3 Recombinant Techniques

Protein and peptides therapeutics according to the present invention can also be obtained by recombinant expression techniques. (See, e.g., Sambrook et al., 1989, Molecular Cloning, A Laboratory Manual, Cold Spring Harbor Laboratory, 2d Ed., Cold Spring Harbor, N.Y., Glover, D. M. (ed.), 1985, DNA Cloning: A Practical Approach, MRL Press, Ltd., Oxford, U.K., Vol. I, II). The nucleic acid sequence encoding human aromatase, testosterone receptor, tryptophan hydroxylase, and neutral amino acid transporter have been cloned and their sequences have been determined. These sequences are known and are available to those practicing in the art. These nucleic acid sequences can be isolated using well-known techniques in the art, such as, for example, screening a library, chemical synthesis, or polymerase chain reaction (PCR).

A gene sequence encoding the respective target protein or peptide is operatively linked to a promoter such that the target protein or peptide is produced from said sequence. For example, a vector can be introduced into a cell, within which cell the vector or a portion thereof is expressed, producing the aromatase protein or peptide. In a preferred embodiment, the nucleic acid is DNA if the source of RNA polymerase is DNA-directed RNA polymerase, but the nucleic acid may also be RNA if the source of polymerase is RNA-directed RNA polymerase or if reverse transcriptase is present in the cell or provided to produce DNA from the RNA. Such a vector can remain episomal or become chromosomally integrated, as long as it can be transcribed to produce the desired RNA. Such vectors can be constructed by recombinant DNA technology methods standard in the art. Vectors can be plasmid, viral, or others known in the art, used for replication and expression in bacterial or mammalian cells. Expression of the sequence encoding the aromatase protein or peptide can be by any promoter known in the art to act in bacterial or mammalian cells. Such promoters can be inducible or constitutive. Such promoters include, but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the HSV-1 (herpes simplex virus-1) thymidine kinase promoter (Wagner et al., 1981, Proc. Natl. Acad. Sci. USA 78:1441-1445), the regulatory sequences of the metallothionein gene (Brinster et al., 1982, Nature 296:39-42), etc., as well as the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in erythroid cells (Mogram et al., 1985, Nature 315:338-340; Kollias et al., 1986, Cell 46, 89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropin releasing hormone gene control region which is active in the hypothalamus (Mason et al., 1986, Science 234:1372-1378). The promoter element which is operatively linked to the nucleic acid encoding the target protein or peptide can also be a bacteriophage promoter with the source of the bacteriophage RNA polymerase expressed from a gene for the RNA polymerase on a separate plasmid, e.g., under the control of an inducible promoter, for example, a nucleic acid encoding the aromatase protein or peptide operatively linked to the T7 RNA polymerase promoter with a separate plasmid encoding the T7 RNA polymerase.

Also included within the scope of the invention are target protein or peptide derivatives which are differentially modified during or after synthesis, e.g., by benzylation, glycosylation, acetylation, phosphorylation, amidation, pegylation, derivatization by known protecting/blocking groups, proteolytic cleavage, linkage to an antibody molecule or other cellular ligand, etc. In specific embodiments, the peptides are acetylated at the N-terminus and/or amidated at the C-terminus. Any of numerous chemical modifications may be carried out by known techniques, including but not limited to acetylation, formylation, oxidation, reduction; metabolic synthesis in the presence of tunicamycin; etc.

In another embodiment, the target protein or peptide derivative is a chimeric, or fusion, protein comprising a functional target protein or peptide joined at its amino- or carboxy-terminus via a peptide bond to an amino acid sequence of a different protein. In one embodiment, such a chimeric protein is produced by recombinant expression of a nucleic acid encoding the chimeric protein (comprising a target protein encoding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

The invention is further defined by reference to the following examples, describing in detail the method of assessing excess androgens in patients presenting with anorexia, AN or a pathologically underweight condition. The following examples also include details of treatment of such disorders with anti-androgens. It will be apparent to those skilled in the art that many modifications, both to materials and methods, may be practiced without departing from the purpose and interest of this invention.

### 5. TREATMENT SUBGROUPS AMONG ANOREXIA NERVOSA PATIENTS

Anorexia nervosa is defined primarily by the fulfillment of certain diagnostic criteria. Classic anorexia nervosa is diagnosed in patients with four distinct signs of anorexia nervosa: (1) severe weight loss in which affected individuals have a body weight less than 85% of expected body weight, (2) an intense fear of weight gain, (3) significant distortion of self-perception of body weight and shape, and (4) amenorrhea (in pre-pubescent females, a delay in the onset of menarche). American Psychiatric Association's Diagnostic & Statistical Manual of Mental Disorders (4th Ed. 1995), § 307.1. Patients with classical anorexia nervosa should be treated aggressively due to the life-threatening nature of the disorder. Frequent follow-up visits should be encouraged and careful monitoring of the patient's progress is imperative.

Not all patients, however, display all four diagnostic signs. Patients that exhibit some, but not all of the signs of anorexia nervosa, however, may be at risk of developing the full spectrum of the disorder. Such patients should be treated early to prevent the devastating sequelae and possible mortality. Recognition of some of the signs of anorexia nervosa may assist the physician in making a diagnosis of a patient who is at risk of developing anorexia nervosa, and thereby prevent the onset of full-blown anorexia nervosa.

### 6. EXAMPLES

### 6.1 Example 1

Physician A examines Patient B, a 19 year old woman brought to the Physician A's office by one of her parents. The patient presents with the following symptoms: emaciation (weighing less than 85% of her expected body weight as determined by comparison to Metropolitan Life insurance tables or pediatric growth charts); fine hairs (lanugo) on her arms and face; and yellowing of the skin (hypercarotenemia). The Physician learns that the woman has not menstruated in five months.

The Physician learns from questioning the Patient that she is a freshman in college and is having a difficult time adjusting. She is also upset over her parents' recent divorce. Physician A then consults with the Patient's mother and learns that the Patient is constantly being told that she is too thin. However, the Patient believes that her legs are too fat, and she is afraid the food at college will “make it worse.”

Physician A diagnoses the Patient B as having AN and prescribes flutamide therapy (initially, 250 mg daily) and asks that the Patient to return for a follow-up visit in one week. She tolerated the therapy well and gains weight. The dose of flutamide is increased to 500 mg daily (250 mg twice a day). She then returns every month for an assessment of her symptomatology using the Yale-Brown-Cornell Eating Disorder Scale (YBC-EDS). Her symptoms improve and her weight increases in the first three months of therapy within the normal range. Her normal menstrual period reappears.

### 6.2 Example 2

Physician C examines Patient D, a 23 year old woman who visits Physician C's office for medical advice concerning irregular menstrual cycles. The Physician learns that Patient D discontinued birth control pills six months ago, but is experiencing irregular periods. The Patient is confident that she is not pregnant. The Physician learns that Patient D broke up with a boyfriend about a year ago and has had trouble dating ever since. The Patient is still upset about the breakup and attributes her trouble in her social life to being “too fat.” On physical examination, the Patient is found to be on the low side of normal for expected body weight, but she is not emaciated. Although Patient D does not fulfill all of the criteria set forth in DSM-IV, the overriding psychological profile of the patient, especially her distorted self-perception and emotional stresses, leads Physician C to make the clinical judgment that the patient may be at an early stage in the process that, untreated, may result in “full-blown” AN. Based on the physician's determination that the patient is therefore at risk for developing AN, and weighing potential complications of therapy, the physician advises the patient of treatment options. The physician recommends flutamide therapy (250 mg daily) and begins oral birth control. The patient returns in one week and is found to have gained weight. She then returns every month for an assessment of her symptomatology using the Yale-Brown-Cornell Eating Disorder Scale (YBC-EDS). Her symptoms improve as she continues to gain weight over the first three months of therapy.

### 6.3 Example 3

Physician E examines Patient F, a 23 year old woman who visits Physician E's office for medical advice concerning irregular menstrual cycles. The Physician learns that Patient F is receiving chemotherapy for cancer of the breast. The patient is nulliparous, and her mother and maternal aunt have breast cancer. Her cancer was detected one year prior to this visit, by routine mammogram, and she had lumpectomy and adjuvant chemotherapy—her last dose one week before the visit. The patient is upset about her weight loss and attributes such loss to the chemotherapeutic treatment. The patient has not received testosterone treatment or tamoxifen (anti-estrogen) treatment. On physical examination, the Patient is found to be on the low side of normal for expected body weight, but she is not emaciated. The physician recommends flutamide therapy (250 mg daily) and begins oral birth control. She returns in one week for an examination and is found to have gained some weight. She then returns every month for follow-up and her weight is recorded. Her symptoms improve and she continues to gains weight over in the first three months of therapy.

### 6.4 Example 4

Physician G examines Patient H, a 66 year old woman who visits Physician G's office for medical advice concerning anorexia and a painful, cold sensation in her extremities, both of which she attributes to chemotherapy she is receiving. The Physician learns that Patient H is receiving 5-fluorouracil (5-FU) and levamisole chemotherapy for cancer of the colon. Her cancer was detected one month prior to this visit, by sigmoidoscopy and she had removal of the tumor by laparoscopic surgery. The tumor was found to be Duke's C. Carcinoembryonic antigen (CEA) was positive before the surgery and became negative when tested three months later. The patient is upset about her weight loss and attributes her to the chemotherapeutic treatment. She feels that she cannot continue with the chemotherapy, despite her oncologist's advice that this treatment has been shown to prolong survival in patients with her condition. On physical examination, the Patient is found to be on the low side of normal for expected body weight, but she is not emaciated. The physician recommends flutamide therapy (250 mg daily). The patient returns in one week for an examination and is found to have gained some weight. She then returns every month for follow-up and her weight is recorded. Her symptoms improve as she continues to gain weight over the first three months of therapy.

### 6.5 Example 5

Physician I examines Patient J, a 25 year old man who visits Physician I's office for medical advice concerning non-intentional weight loss. The Physician learns that Patient J has been given the diagnosis of Acquired Immune Deficiency Syndrome (AIDS) and has signs, symptoms and laboratory tests consistent with the diagnosis of AIDS. The patient was first diagnosed with HIV-1 infection when pneumocystis pneumonia was diagnosed by fiberoptic brochoscopy. The patient's risk factors for HIV-1 infection include unprotected sex with prostitutes and occasional intravenous drug abuse. The patient is well except for memory loss and problems at work that suggested a moderate cognitive dysfunction. The patient is taking anti-viral therapy regularly, but continues to lose weight. On physical examination, the Patient is found to be on the low side of normal for expected body weight. He has sunken eyes and hollowed temples (temporal wasting), indicating significant cachexia. The physician recommends flutamide therapy (750 mg daily, given in three divided doses). The patient is asked to monitor his weight at home and to return for a follow-up visit in one week. The therapy is tolerated well, after initial problems with diarrhea and breast tenderness. The patient gains weight over the next three months, during which time he is evaluated every three weeks.

### 6.6 Example 6

Physician K examines Patient L, a 23 year old man who visits Physician K's office for medical advice concerning weight loss. The Physician learns that Patient L takes intravenous heroin and has been unable to discontinue this habit despite inpatient detox. The patient works and has a family, so he is afraid that weight loss will be detected at work and result in a re-evaluation of his job performance and employer-mandated substance abuse rehabilitation/maintenance program that includes scheduled drug tests, but allows for his supervisor to request random drug testing upon suspicion of recidivism/recurrence. The Patient expresses the view that the weight loss plays a significant role in driving him to continue to use heroin, which he feels relieves fears about his physical appearance. On physical examination, the Patient is found to be on the low side of normal for expected body weight, and to have sunken eyes and hollowed temples, suggesting significant cachexia. Except for “track marks” from needle injections, and lymphadenopathy the physical exam is otherwise unremarkable. The patient has a reactive skin test to candida and a negative PPD test. Although by history the patient describes a positive HIV test in the past, repeat ELISA is negative and T4 cells are 650/cc. Chest X-ray is unremarkable, skeletal survey is negative, carcinoembryonic antigen (CEA), alpha-fetal protein (AFP) and Prostate Specific Antigen (PSA) are negative and the serum protein electrophoresis (SPEP) shows polyclonal or “diffuse” hypergammaglobulinemia. The physician makes the provisional diagnosis of cachexia secondary to intravenous drug abuse. The physician recommends flutamide therapy (750 mg daily, given in three divided doses). The patient is asked to monitor his weight at home and to return for a follow-up visit in one week. The patient continues to gain weight over the next three months, during which time he is evaluated every three weeks.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing examples. Such modifications are intended to fall within the scope of the appended claims.

1. A method for treating or preventing anorexia nervosa comprising administering a therapeutically effective amount of an anti-androgen.

2. The method of claim 1 wherein said anti-androgen is an androgen synthesis inhibitor.

3. The method of claim 1 wherein said anti-androgen is a promoter of androgen metabolism.

4. The method of claim 1 wherein said anti-androgen is an inhibitor that interferes with the binding of natural androgen to androgen receptor.

5. The method of claim 1 wherein said anti-androgen is an inhibitor that interferes with androgen receptor signaling.

6. The method of claim 2 wherein said androgen synthesis inhibitor is spironolactone, ketoconozole, ciprofloxacin, lovastatin, simvastatin, finasteride, epitestosterone, PCE 28260, FCE 28175, or FCE 27837.

7. The method of claim 3 wherein said promoter of androgen metabolism is aromatase.

8. The method of claim 4 wherein said inhibitor is flutamide, nilutamide, zanoterone, casodex, cyproterone, acetate, pyrethroids, Anandron (RU 23908) or other non-steroidal anti-androgens (RU 59063, RU 56187).

9. The method of claim 4 wherein said inhibitor is flutamide.

10. The method of claim 9 wherein the flutamide is administered in a dosage of 250-750 mg per day.

11. A method for treating a pathologically underweight human comprising administering to said human a therapeutically effective amount of an anti-androgen.

12. The method according to claim 11, wherein said pathologically underweight condition is a feature of drug addiction or drug withdrawal.

13. The method of claim 11 wherein said anti-androgen is an androgen synthesis inhibitor.

14. The method of claim 11 wherein said anti-androgen is a promoter of androgen metabolism.

15. The method of claim 11 wherein said anti-androgen is an inhibitor that interferes with binding of natural androgen to androgen receptor.

16. The method of claim 11 wherein said anti-androgen is an inhibitor that interferes with androgen receptor signaling.

17. The method of claim 13 wherein said androgen synthesis inhibitor is spironolactone, ketoconozole, ciprofloxacin, lovastatin, simvastatin, finasteride, epitestosterone, FCE 28260, FCE 28175 or FCE 27837.

18. The method of claim 14 wherein said promoter of androgen metabolism is aromatase.

19. The method of claim 15 wherein said inhibitor is flutamide, nilutamide, zanoterone, casodex, cyproterone acetate, pyrethroids, Anandron (RU 23908) or other non-steroidal anti-androgens (RU 59063, RU 56187).

20. The method of claim 15 wherein said inhibitor is flutamide.

21. The method of claim 20 wherein the flutamide is administered at a dosage of 250-750 mg per day.

22. The method of claim 1 or 11 further comprising the concurrent administration of oral birth control compounds.

23. The method of claim 1 or 11 further comprising the use of the carrier DHA or RMP-7.

24. The method according to claim 1 or 11 wherein said anti-androgen administered is the recombinant protein aromatase, tryptophan hydroxylase, neutral amino acid transporter, or inactive testosterone receptor.

25. The method of claim 24 wherein said recombinant protein is a fusion protein with a protein that enables penetration of the blood-brain barrier.

26. The method of claim 25, wherein said fusion protein contains a peptide sequence of nerve growth factor or transthyrotein.

27. The method of claim 24 wherein said recombinant protein is translated from a recombinant expression vector administered to a patient's somatic cells.

28. The method of claim 27 wherein said recombinant protein is neutral amino acid transporter.

29. The method according to claim 1 or 11 wherein said anti-androgen administered is an inhibitory testosterone receptor.
